(19) United States
(12) Patent Application Publication
(10) Pub. No.: US 2023/0374030 A1
(43) Pub. Date: Nov. 23, 2023

(54) SOLID-STATE FORMS OF RELUGOLIX

(71) Applicant: MACFARLAN SMITH LIMITED, Edinburgh (GB)

(72) Inventor: Nicholas PASCHALIDES, Devens, MA (US)

(21) Appl. No.: 17/597,998

(22) PCT Filed: Jul. 31, 2020
(86) PCT No.: PCT/US2020/044553

§ 371 Date: Feb. 1, 2022

Related U.S. Application Data
(60) Provisional application No. 62/882,297, filed on Aug. 2, 2019.

(51) Int. Cl. C07D 495/04

(52) U.S. Cl. CPC C07D 495/04

(57) ABSTRACT

The present invention is directed to a solid-state DMF solvate of relugolix and to solid-state anhydrous forms of relugolix designated as Form A and Form C of anhydrous relugolix. The present invention is further directed to processes for the preparation of the solid-state DMF solvate of relugolix and each of Form A, Form B, and Form C of anhydrous relugolix. The present invention also is directed to pharmaceutical compositions comprising the DMF solvate of relugolix or Form A or Form C of anhydrous relugolix, and to a method for treating disease using the DMF solvate of relugolix or Form A or Form C of anhydrous relugolix.

### FIELD OF THE INVENTION

The invention relates to a solid-state DMF solvate and anhydrous forms of relugolix and to methods for their preparation. The present disclosure also relates to pharmaceutical compositions comprising the novel forms of relugolix and methods for treating disease using the forms.

### BACKGROUND OF THE INVENTION

Relugolix, having the chemical designation, 1-[4-[1-[(2,6-difluorophenyl)-methyl]-5-[(dimethylamino)methyl]-3-(6-methoxypyridazin-3-yl)-2,4-dioxothieno-[2,3-d]pyrimidin-6-yl]phenyl]-3-methoxyurea, is an orally active nonpeptide gonadotropin-releasing hormone (GnRH)-receptor antagonist. Relugolix has the following structure:

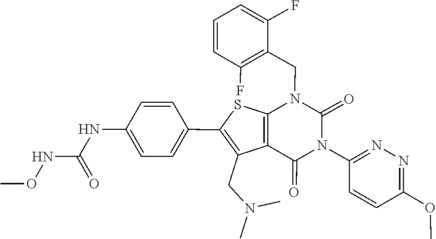

Relugolix has been approved in Japan as a treatment for symptoms associated with uterine fibroids. Studies are on-going to evaluate the efficacy of relugolix as a treatment for endometriosis-associated pain and prostate cancer.

U.S. Pat. No. 10,464,945 discloses a crystalline form of a tetrahydrofuran solvate of relugolix, and another crystalline form that exhibits an x-ray powder diffraction pattern having 2-theta (2θ) peaks at approximately 8.932°, 16.607°, and 17.328°. Other XRPD peaks include approximately 7.384°, 9.933°, 12.076°, 22.202°, 22.761°, and 27.422° 2θ.

WO2019/178304 discloses several forms of relugolix. Specifically, Form F is described as an isostructural polymorph, i.e., it may be either anhydrous, a hydrate, preferably a hemi-hydrate, or a solvate. It is characterized by an X-ray powder diffraction pattern having peaks at 6.9, 7.5, 9.5, 13.9 and 18.1° 2θ±0.2° 2θ. Form G is characterized by an X-ray powder diffraction pattern having peaks at 5.4, 8.4, 10.7 and 12.1° 2θ±0.2° 2θ. Polymorphically pure Form G is characterized by an X-ray powder diffraction pattern having peaks at 3.4, 5.6, 9.6, 13.3 and 17.4° 2θ±0.2° 2θ. Form H is characterized by an X-ray powder diffraction pattern having peaks at 6.2, 8.6, 15.9, 19.0 and 19.6° 2θ±0.2° 2θ. Form J is described as a hemi acetonitrile solvate, hemihydrate. WO2019/178304 also discloses an amorphous form of relugolix.

There is no disclosure of a DMF solvate of relugolix, more particularly having at least 2 or more X-ray powder diffraction peaks selected from about 20.1, 24.3 and 9.0° 2θ, or anhydrous crystalline forms of relugolix having X-ray powder diffraction peaks selected from either about 10.7, 20.9 and 19.2° 2θ or about 8.3, 6.8, 7.7, and 19.9° 2θ.

### SUMMARY OF THE DISCLOSURE

The present invention is directed to a solid-state DMF solvate of relugolix, designated as Form A of the DMF solvate of relugolix, and to solid-state anhydrous forms of relugolix, designated as Form A and Form C of anhydrous relugolix. The present invention is further directed to processes for the preparation of Form A of the DMF solvate of relugolix and each of Form A, Form B, and Form C of anhydrous relugolix. The present invention also is directed to pharmaceutical compositions comprising Form A of the DMF solvate of relugolix or either Form A or Form C of anhydrous relugolix, and to a method for treating disease using Form A of the DMF solvate of relugolix or either Form A or Form C of anhydrous relugolix.

### BRIEF DESCRIPTION OF THE DRAWINGS

FIG. 1 provides an overlay of a calculated XRPD pattern from a single crystal of Form A of the DMF solvate of relugolix (bottom) and actual XRPD pattern of Form A of the DMF solvate of relugolix (top).

FIG. 2 provides a three-dimensional structure of Form A of the DMF solvate of relugolix that is discerned from SCXRD.

FIG. 3 provides a representative DSC plot of Form A of the DMF solvate of relugolix.

FIG. 4 provides a representative TGA plot of Form A of the DMF solvate of relugolix.

FIG. 5 provides a representative DVS plot of Form A of the DMF solvate of relugolix.

FIG. 6 provides a representative ¹H-NMR plot of Form A of the DMF solvate of relugolix.

FIG. 7 provides a representative XRPD pattern of Form A of anhydrous relugolix.

FIG. 8 provides a representative DSC plot of Form A of anhydrous relugolix.

FIG. 9 provides a representative TGA plot of Form A of anhydrous relugolix.

FIG. 10 provides a representative DVS plot of Form A of anhydrous relugolix.

FIG. 11 provides a representative ¹H-NMR plot of Form A of anhydrous relugolix.

FIG. 12 provides a representative XRPD pattern of Form B of anhydrous relugolix.

FIG. 13 provides a representative DSC plot of Form B of anhydrous relugolix.

FIG. 14 provides a representative TGA plot of Form B of anhydrous relugolix.

FIG. 15 provides a representative DVS plot of Form B of anhydrous relugolix.

FIG. 16 provides a representative ¹H-NMR plot of Form B of anhydrous relugolix.

FIG. 17 provides a representative XRPD pattern of Form C of anhydrous relugolix.

FIG. 18 provides a representative DSC plot of Form C of anhydrous relugolix.

FIG. 19 provides a representative TGA plot of Form C of anhydrous relugolix.

FIG. 20 provides a representative DVS plot of Form C of anhydrous relugolix.

FIG. 21 provides a representative ¹H-NMR plot of Form C of anhydrous relugolix.

### DETAILED DESCRIPTION OF THE DISCLOSURE

The present disclosure is directed to a solid-state DMF solvate of relugolix, designated as Form A of the DMF solvate of relugolix, and to anhydrous forms of relugolix, designated as Form A and Form C of anhydrous relugolix; pharmaceutical compositions comprising Form A of the DMF solvate of relugolix or either Form A or Form C of anhydrous relugolix; processes for the preparation of Form A of the DMF solvate of relugolix and each of Form A, Form B, and Form C of anhydrous relugolix; and the use of Form A of the DMF solvate of relugolix or either Form A or Form C of anhydrous relugolix for treating a patient with uterine fibroids, endometriosis, or prostate cancer.

As used herein and unless otherwise specified, the term “solid-state form” includes crystalline or polymorphic forms, amorphous phase, and solvates.

As used herein and unless otherwise specified, the terms “about” and “approximately,” when used in connection with a numeric value or a range of values which is provided to characterize a particular solid form, e.g., a specific temperature or temperature range, such as, e.g., that describing a DSC or TGA thermal event, including, e.g., melting, dehydration, desolvation or glass transition events; a mass change, such as, e.g., a mass change as a function of temperature or humidity; a solvent or water content, in terms of, e.g., mass or a percentage; or a peak position, such as, e.g., in analysis by IR or Raman spectroscopy or XRPD; indicate that the value or range of values may deviate to an extent deemed reasonable to one of ordinary skill in the art while still describing the particular solid form.

As used herein and unless otherwise specified, the term “pharmaceutical composition” is intended to encompass a pharmaceutically effective amount of Form A of the DMF solvate of relugolix, or either Form A or Form C of anhydrous relugolix and a pharmaceutically acceptable excipient. As used herein, the term “pharmaceutical compositions” includes pharmaceutical compositions such as tablets, pills, powders, liquids, suspensions, emulsions, granules, capsules, suppositories, or injection preparations.

As used herein and unless otherwise specified, the term “crystalline” and related terms used herein, when used to describe a compound, substance, modification, material, component or product, unless otherwise specified, mean that the compound, substance, modification, material, component or product is substantially crystalline as determined by X-ray diffraction. See, e.g., Remington: The Science and Practice of Pharmacy, 21st edition, Lippincott, Williams and Wilkins, Baltimore, Md. (2005); The United States Pharmacopeia, 23rd ed., 1843-1844 (1995).

As used herein and unless otherwise specified, the term “excipient” refers to a pharmaceutically acceptable organic or inorganic carrier substance. Excipients may be natural or synthetic substances formulated alongside the active ingredient of a medication, included for the purpose of bulking-up formulations that contain potent active ingredients (thus often referred to as “bulking agents,” “fillers,” or “diluents”), or to confer a therapeutic enhancement on the active ingredient in the final dosage form, such as facilitating drug absorption or solubility. Excipients can also be useful in the manufacturing process, to aid in the handling of the active substance, such as by facilitating powder flowability or non-stick properties, in addition to aiding in vitro stability such as prevention of denaturation over the expected shelf life.

As used herein and unless otherwise specified, the term “patient” refers to an animal, preferably a mammal, most preferably a human, who has been the object of treatment, observation or experiment. Preferably, the patient has experienced and/or exhibited at least one symptom of the disease or disorder to be treated and/or prevented. Further, a patient may not have exhibited any symptoms of the disorder, disease or condition to be treated and/or prevented, but has been deemed by a physician, clinician or other medical professional to be at risk for developing said disorder, disease or condition.

As used herein and unless otherwise specified, the terms “polymorph,” “polymorphic form” or related term herein, refer to a crystal form of an API (active pharmaceutical ingredient) free base or salt thereof that can exist in two or more forms, as a result of different arrangements or conformations of the molecule, ions of the salt, or addition and arrangement of solvents within the crystalline lattice.

As used herein and unless otherwise specified, the terms “substantially” or “substantially free/pure” with respect to a polymorph or polymorphic form means that the form contains about less than 30 percent, about less than 20 percent, about less than 15 percent, about less than 10 percent, about less than 5 percent, or about less than 1 percent by weight of impurities. Impurities may, for example, include other polymorphic forms, water and solvents other than that in a solvated crystalline polymorphic form.

As used herein and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more therapeutic agents to a patient with such a disease or disorder. In some embodiments, the terms refer to the administration of a compound provided herein, with or without other additional active agents, after the onset of symptoms of the particular disease.

As used herein and unless otherwise specified, the abbreviation “DMF” refers to dimethylformamide; the abbreviation “TBME” refers to tert-butylmethyl ether; the abbreviation “DCM” refers to dichloromethane; and the abbreviation “IPAc” refers to isopropyl acetate.

An object of the present disclosure is directed to Form A of the DMF solvate of relugolix and solid-state anhydrous forms of relugolix, designated as Form A and Form C of anhydrous relugolix, that are substantially pure, stable and scalable. It is also an object of the present disclosure to provide Form A of the DMF solvate of relugolix and solid-state anhydrous forms of relugolix, designated as Form A and Form C of anhydrous relugolix, that are capable of being isolated and handled. It is further an object of the present disclosure to provide processes for the preparation of Form A of the DMF solvate of relugolix and each of Form A, Form B, and Form C of anhydrous relugolix. It is yet another object of the present disclosure to provide a method of use of Form A of the DMF solvate of relugolix and Form A and Form C of anhydrous relugolix to prepare a pharmaceutical dosage form of relugolix.

Techniques for characterizing crystal and amorphous forms include but are not limited to differential scanning calorimetry (DSC), thermal gravimetric analysis (TGA), dynamic vapor sorption (DVS), X-ray powder diffractometry (XRPD), single crystal X-ray diffraction (SCXRD), proton nuclear magnetic resonance (¹H-NMR), Fourier transform infrared spectroscopy (FTIR Spectroscopy), and Optical Microscopy.

TGA data are collected using a TA Instruments TGA Q500. Samples (about 2-5 mg) are placed in a pin holed sealed hermetic alodined aluminum DSC pan, pre-tared with an aluminum pan and scanned from about 30 to about 300° C. at a rate of about 10° C./min using a nitrogen purge at about 60 mL/min.

X-ray powder diffraction patterns are obtained using a Bruker D8 Advance equipped with a Cu Kα radiation source (λ=1.54° A), a 9-position sample holder and a LYNXEYE super speed detector. Samples are placed on air sensitive silicon plate holders with zero-background with domes, for analysis. One skilled in the art would recognize that the °2θ values and the relative intensity values are generated by performing a peak search on the measured data and that the d-spacing values can be calculated by the instrument from the °2θ values using Bragg's equation. One skilled in the art would further recognize that the relative intensity for the measured peaks may vary as a result of sample preparation, orientation and instrument used, for example.

The X-ray intensity data for SCXRD are collected on a Bruker D8QUEST [1] CMOS area detector employing graphite-monochromated Mo-Kα radiation (λ=0.71073Å) at a temperature of 100 K.

DVS samples are analyzed using a TA Instruments Q5000SA gravimetric water sorption analyzer. The relative humidity is adjusted between about 0-95% and the weight of the sample is continuously monitored and recorded with respect to the relative humidity and time.

DSC data are collected using a TA Instruments Q10 DSC. About 2-8 mg of sample are placed in sealed but covered hermetic alodined aluminum sample pan and scanned from about 30 to about 300° C. at a rate of about 10° C./min under a nitrogen purge of about 50 mL/min. Additionally, DSC runs are generated on a TA Instruments Q2000 equipped with an auto-sampler and RSC40. The instrument is programmed with about a 10° C./min ramp rate from about 25° C. to about 300° C. using Tzero hermetically sealed aluminum pans in T4P (or T4) mode.

¹H NMR samples are prepared by dissolving the compound in deuterated dimethylsulfoxide and deuterated chloroform with about 0.05% (v/v) tetramethylsilane (TMS). Spectra are collected at ambient temperature on a Bruker Avance 600 MHz NMR equipped with TopSpin software. The number of scans is 16 for ¹H-NMR at 298 K.

In one embodiment, Form A of the DMF solvate of relugolix is prepared by:
a) mixing a solution of relugolix in DMF with an anti-solvent; and
b) stirring the mixture of step a) to yield Form A of the DMF solvate of relugolix as a precipitate.
   In one embodiment, the ratio of relugolix to DMF in the solution of relugolix in DMF is about 1:5 weight (g_{relugolix}) to volume (mL_{DMF}). In a particular embodiment, the anti-solvent is TBME. In another embodiment the anti-solvent is toluene. It will be apparent to one of ordinary skill in the art that other anti-solvents, such as, for example but without being limited to, heptane, xylene, or cumene, can be used depending on their anti-solvent properties. In one embodiment, about 10-13 volumes of anti-solvent is mixed with the solution of relugolix in DMF (weight (g_{relugolix}) to volume(mLₐₙₜᵢ₋ₛₒₗᵥₑₙₜ)). In one embodiment, the anti-solvent is added to the solution of relugolix in DMF. In a particular embodiment, the precipitation occurs at ambient temperature. Another embodiment further comprises reducing the temperature of the mixture of the solution of relugolix in DMF and anti-solvent to the nucleation temperature for about 30 minutes to 1 hour to produce a precipitate. The nucleation temperature is readily determined by one of ordinary skill in the art. The temperature is slowly lowered from the nucleation temperature about 2-5° C. per minute to about 5° C. Another embodiment is wherein larger particles of relugolix are produced. In one embodiment, the stirring occurs for about 15-18 hours. In other embodiments, the stirring occurs for a shorter period of time. Another embodiment further comprises isolating the precipitate. Another embodiment further comprises using additional anti-solvent to facilitate the isolating of the precipitate. Another embodiment further comprises using additional anti-solvent to wash the precipitate. In one embodiment, the isolating is effected by vacuum filtration. One embodiment further comprises drying the precipitate. In one embodiment, the drying is under vacuum at about 45° C. In one embodiment, the drying occurs for at least about 8 hours to overnight (about 16-24 h). Another embodiment further comprises preparing the solution of relugolix in DMF by dissolving relugolix in DMF. In one embodiment, the relugolix is dissolved in DMF at ambient temperature. In another embodiment, heat is applied to facilitate the dissolution. Another embodiment further comprises preparing the solution of relugolix in DMF by combining relugolix and DMF, wherein the relugolix is formed by a chemical reaction in solution, for example, by deprotection. It will be apparent to one of ordinary skill in the art that any relugolix may be used, regardless of its solid-state form, in the solution of relugolix in DMF. Depending on the purity of the relugolix, it may be necessary or desirable to remove any or all unwanted salts from the relugolix by water extractions or to remove any or all other impurities before preparing the solution of relugolix in DMF.

In another embodiment, Form A of anhydrous relugolix is prepared by
a) forming a solution of relugolix in acetone wherein the relugolix is in about 10 volumes of acetone (weight(g_{relugolix}): volume(mL_{acetone})); and
b) stirring the solution of relugolix in acetone to yield Form A of anhydrous relugolix as a precipitate.
   In one embodiment, the stirring occurs for about 5-10 minutes. An embodiment is wherein the forming the solution of relugolix in acetone is by dissolving relugolix in acetone. In another embodiment, the forming the solution of relugolix in acetone is by combining relugolix and acetone, wherein the relugolix is formed by a chemical reaction in solution, for example, by deprotection. It will again be apparent to one of ordinary skill in the art that any relugolix may be used for forming the solution of relugolix in acetone, regardless of its solid-state form and that it may be desirable to remove any or all unwanted salts by water extractions or to remove any or all other impurities prior to forming the solution of relugolix in acetone. Another embodiment further comprises isolating the precipitate.

In another embodiment, Form B of anhydrous relugolix is prepared by
a) forming a solution of relugolix in DCM wherein the relugolix is in about 20 volumes of DCM (weight(g_{relugolix}):volume(mL_{DCM})); and
b) evaporating the DCM to yield Form B of anhydrous relugolix.
   One embodiment further comprises preparing the solution of relugolix in DCM by dissolving relugolix in DCM. Another embodiment further comprises preparing the solution of relugolix in DCM by combining relugolix and DCM, wherein the relugolix is formed by a chemical reaction in solution, for example, by deprotection. In another embodiment the evaporating the DCM is carried out with a rotary evaporator at about 35° C. and under a high vacuum pump for at least about 3 hours. It will be apparent to one of ordinary skill in the art that any relugolix may be used, regardless of its solid-state form, in the solution of relugolix in DCM. Depending on the purity of the relugolix, it may be necessary or desirable to remove any or all unwanted salts from the relugolix by water extractions or to remove any or all other impurities before preparing the solution of relugolix in DCM.

In another embodiment, Form B of anhydrous relugolix is prepared by
a) mixing a solution of relugolix in DCM wherein the relugolix is in at least about 20 volumes of DCM (weight(g_{relugolix}):volume(mL_{DCM}) with an anti-solvent wherein the anti-solvent is at about a 1:1 ratio of anti-solvent to DCM (volumeₐₙₜᵢ₋ₛₒₗᵥₑₙₜ:volume_{DCM});
b) stirring the mixture of step a) for a period of time to yield Form B of anhydrous relugolix as a precipitate.
   One embodiment further comprises preparing the solution of relugolix in DCM by dissolving relugolix in DCM. Another embodiment further comprises preparing the solution of relugolix in DCM by combining relugolix and DCM, wherein the relugolix is formed by a chemical reaction in solution, for example, by deprotection. In one embodiment, the stirring occurs overnight (about 16-24 h). One embodiment further comprises concentrating the solution of relugolix in DCM to a certain volume before mixing with the anti-solvent. In various embodiments, the anti-solvent is cumene, cyclohexane, TBME, heptane, or toluene. It will be apparent to one of ordinary skill in the art that any relugolix may be used, regardless of its solid-state form, in the solution of relugolix in DCM. Depending on the purity of the relugolix, it may be necessary or desirable to remove any or all unwanted salts from the relugolix by water extractions or to remove any or all other impurities before preparing the solution of relugolix in DCM. Another embodiment further comprises isolating the precipitate.

In another embodiment, Form C of anhydrous relugolix is prepared by
a) adding about 10 volumes of an organic solvent to Form B of anhydrous relugolix (weight(g_{relugolix}):volume(mL_{organic solvent}); and
b) stirring the mixture of organic solvent and Form B of anhydrous relugolix overnight (about 16-24 h) resulting in a slurry of Form C of anhydrous relugolix.

In one embodiment, the organic solvent is isopropyl acetate or 2-butanol. One embodiment further comprises drying Form C of anhydrous relugolix in a vacuum oven at about 35-40° C. overnight (about 16-24 h). Another embodiment further comprises isolating Form C of anhydrous relugolix from the slurry, for example by decanting or filtering.

The present disclosure also encompasses a pharmaceutical composition comprising Form A of the DMF solvate of relugolix or Form A or Form C of anhydrous relugolix and a pharmaceutically acceptable excipient. A pharmaceutical composition containing Form A of the DMF solvate of relugolix or Form A or Form C of anhydrous relugolix may be prepared according to U.S. Pat. No. 10,350,170, U.S. Patent Application Publication No. 2011/0172249, or any other methods known in the art.

The present disclosure provides for a method of treating disease by administering to a patient, in need thereof, a pharmaceutical composition comprising Form A of the DMF solvate of relugolix or Form A or Form C of anhydrous relugolix. Relugolix has been approved for the treatment of uterine fibroids in Japan and may also be used in the treatment of endometriosis and prostate cancer. It may be used in combination with one or more pharmaceutically acceptable agents, for example, low-dose estradiol and norethindrone acetate.

The dosage of the pharmaceutical compositions may be varied over a wide range. Optimal dosages and dosage regimens to be administered may be readily determined by those skilled in the art, and will vary with the mode of administration, the strength of the preparation and the advancement of the disease condition. In addition, factors associated with the particular patient being treated, including patient's sex, age, weight, diet, physical activity, time of administration and concomitant diseases, will result in the need to adjust dosages and/or regimens.

### EXAMPLES

Examples 1-4, which follow herein, provide embodiments of the preparation of Form A of the DMF solvate of relugolix and each of Form A, Form B, and Form C of anhydrous relugolix.

The Examples are presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles described herein may be applied to other examples and applications without departing from the spirit and scope of the various embodiments. Therefore, the various embodiments are illustrative of the present disclosure and the disclosure is not intended to be limited to the examples described herein and shown.

### Example 1

### Preparation of Form A of the DMF Solvate of Relugolix

1.63 g of Form B of anhydrous relugolix is dissolved with DMF (7.6 g, 8.2 mL). The total solution weight (solvent+API) is 9.2 g. From that solution, equal amounts are transferred into two 100 mL RB (round bottom) Flasks equipped with the same size/shape magnetic stirring bar at the same agitation speed (about 700 RPMs); about 4.37 g of DMF/API solution (about 775 mg of API) is contained in each flask.

10.2 mL of TBME (about 13 volumes TBME (mL) to weight of API (g)) is added to one flask and 10.2 mL of toluene (about 13 volumes toluene (mL) to weight of API (g)) is added to the second flask. The contents of each flask are stirred. Signs of precipitation are shown within the first 10 minutes of agitation in the TBME flask. Signs of precipitation are shown the following day in the toluene flask.

The contents of each flask are separately vacuum filtered using a Buckner funnel with paper filter. Additional TBME (2×4 mL) is used to transfer all the material in the TBME flask onto the filter. The isolated material is dried under vacuum at about 45° C. for about 8 hours. 770 mg (89.5% isolated yield) of Form A of the DMF solvate of relugolix is obtained as a yellow solid and having a 1:1 API to DMF solvent ratio.

No additional toluene is required to transfer the material from the toluene flask onto the filter. The isolated material is dried under vacuum at about 45° C. for about 8 hours. 694 mg (80.3% isolated yield) of Form A of the DMF solvate of relugolix is obtained as a yellow solid and having a 1:1 API to DMF solvent ratio.

Form A of the DMF solvate of relugolix is stable, i.e., it is unchanged after prolonged drying (e.g., about 2 days) under vacuum at about 70° C. It also remains unchanged under about 97% humidity at ambient temperatures for over a month.

XRPD 2θ pattern peaks and relative % intensity values for the peaks of Form A of the DMF solvate of relugolix are shown in Table I.

TABLE 1
Average Peak List for Form A
of the DMF solvate of
relugolix diffractogram

| Angle (2θ°) | Intensity |
|---|---|
| degree | % |
| 8.6 | 16.7 |
| 9.0 | 40.4 |
| 11.0 | 14.1 |
| 12.8 | 30.2 |
| 13.2 | 16.5 |
| 13.5 | 13.3 |
| 13.8 | 13.3 |
| 14.4 | 12.7 |
| 18.2 | 29.4 |
| 19.1 | 32.5 |
| 20.1 | 100 |
| 21.7 | 29 |
| 24.3 | 57.5 |
| 25.4 | 25.1 |
| 25.9 | 39.6 |
| 30.9 | 24.3 |

The angle measurements are ±0.2° 2θ. Key defining peaks for solid-state Form A of the DMF solvate of relugolix include two or more of 20.1, 24.3, and 9.0° 2θ.

Single crystal parameters for Form A of the DMF solvate of relugolix as determined by SCXRD are:
Crystal System: Triclinic Space Group P1
a=11.1Å±1.5%
b=12.0Å±1.5%
c=14.0Å±1.5%
α=112°±3°
β=110°±3°
γ=91°±3°
Cell Volume: 1609Å³±3%

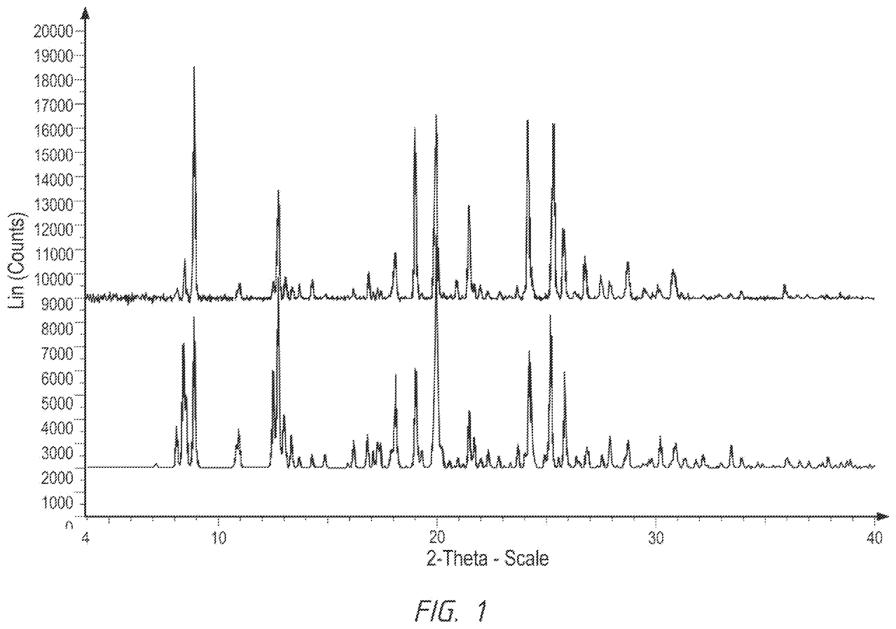

An XPRD pattern for a representative sample of Form A of the DMF solvate of relugolix (top) and a calculated XRPD pattern from a single crystal of Form A of the DMF solvate of relugolix (bottom) are shown in FIG. 1.

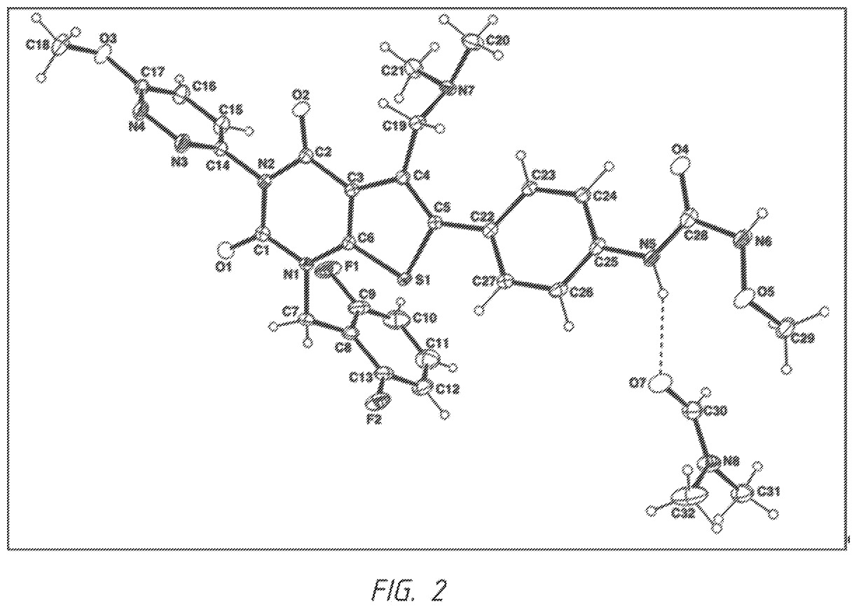

A three-dimensional structure of Form A of the DMF solvate of relugolix that is discerned from SCXRD is shown in FIG. 2.

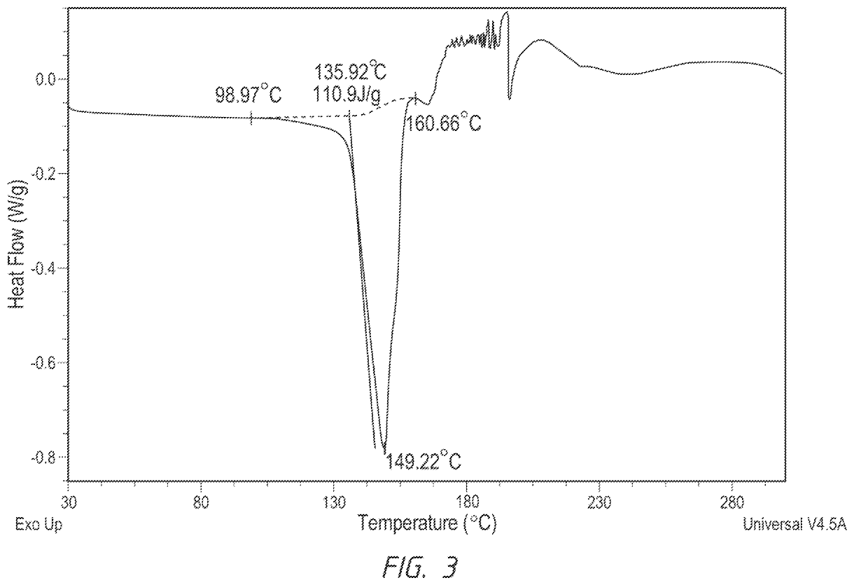

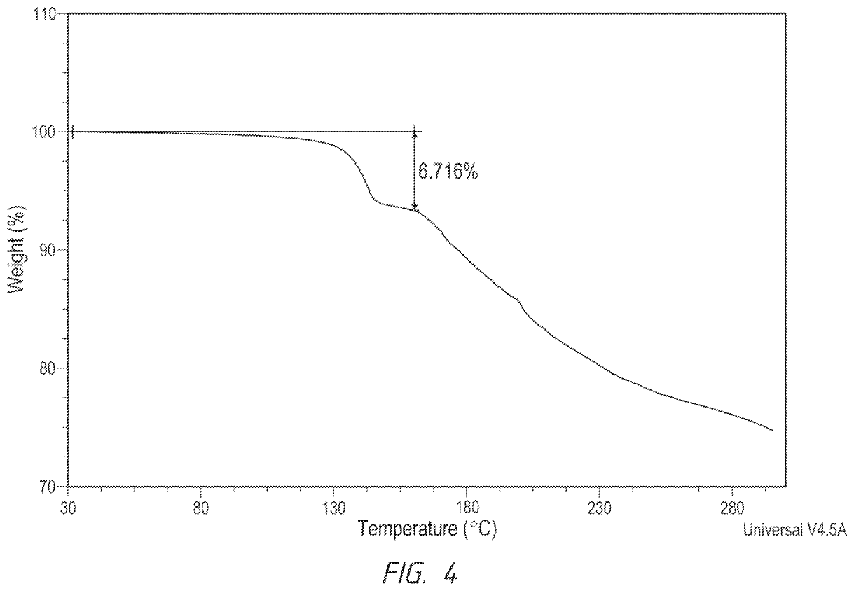

DSC analysis of Form A of the DMF solvate of relugolix shows the onset of an endothermic event at about 99° C. and a sharp endothermic event at about 149° C., as depicted in FIG. 3, and TGA analysis shows a loss of about 6.7 weight % up to about 155° C., as depicted in FIG. 4.

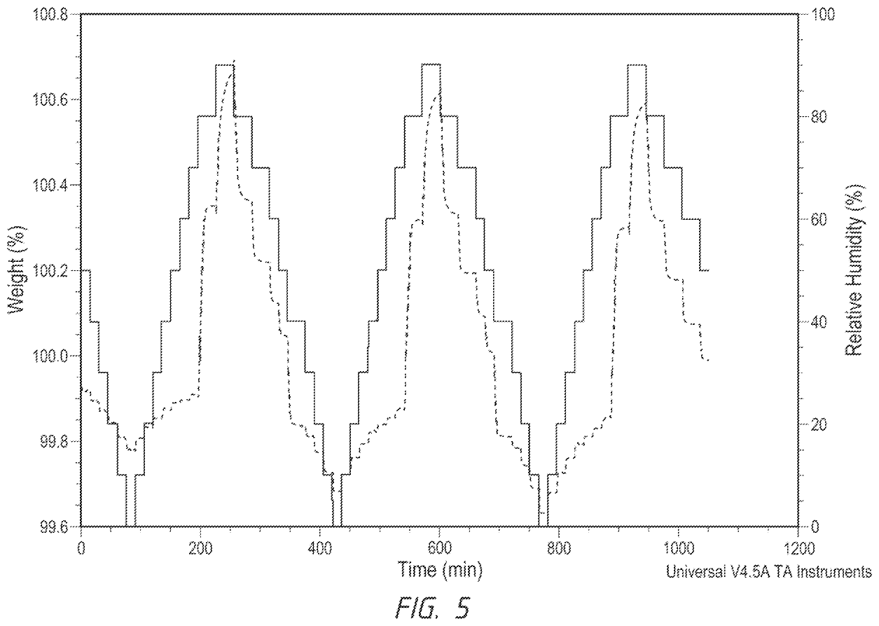

A representative DVS plot of Form A of the DMF solvate of relugolix indicates the loss of about 1% mass at about 90% RH as depicted in FIG. 5.

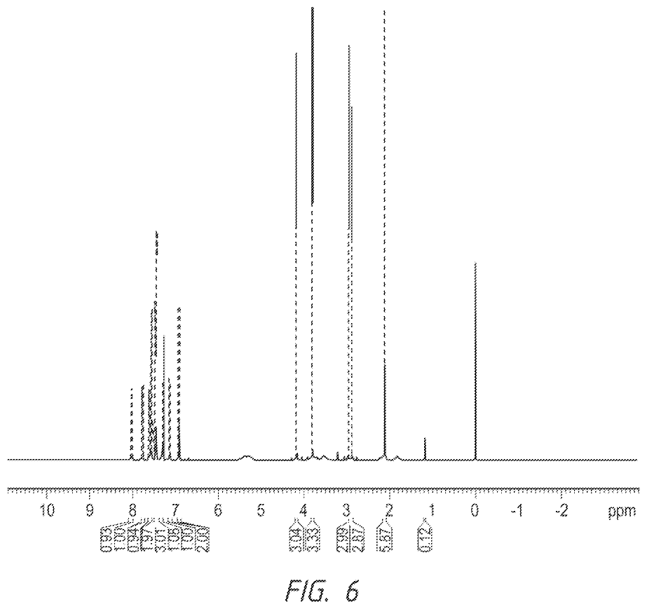

¹H NMR analysis indicates the presence of DMF in Form A of the DMF solvate of relugolix, as depicted in FIG. 6.

### Example 2

### Preparation of Form A of Anhydrous Relugolix

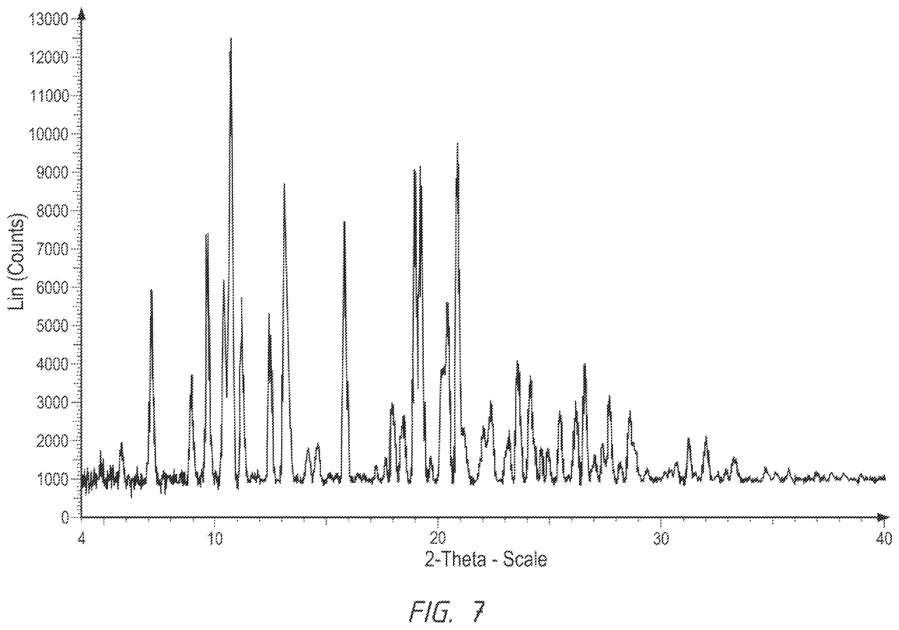

Form B of anhydrous relugolix is dissolved in about 10 volumes of acetone (weight(g_{relugolix}):volume(mL_{acetone}). The solution is stirred and re-crystalizes in about 5 to 10 minutes as Form A of anhydrous relugolix, as evidenced by its XRPD pattern contained in FIG. 7.

XRPD 2θ pattern peaks and relative % intensity values for the peaks of Form A of anhydrous relugolix are shown in Table 2.

TABLE 2
Average Peak List for Form A of
anhydrous relugolix diffractogram

| Angle (2θ°) | Intensity |
|---|---|
| degree | % |
| 5.8 | 15.7 |
| 7.1 | 48.1 |
| 8.9 | 29.9 |
| 9.6 | 59.6 |
| 10.4 | 49.1 |
| 10.7 | 100 |
| 11.2 | 46 |
| 12.4 | 42.4 |
| 13.1 | 69.9 |
| 14.1 | 14.7 |
| 14.5 | 14.7 |
| 15.8 | 62.2 |
| 17.6 | 12.7 |
| 17.9 | 24.1 |
| 18.5 | 21.3 |
| 18.9 | 72.5 |
| 19.2 | 72.7 |
| 20.4 | 44.4 |
| 20.9 | 78.2 |
| 21.1 | 19 |
| 22.0 | 19.1 |
| 22.4 | 24.8 |
| 23.2 | 18.1 |
| 23.5 | 33 |
| 24.2 | 29.6 |
| 24.6 | 14.4 |
| 24.9 | 13.9 |
| 25.5 | 22.1 |
| 26.2 | 24.5 |
| 26.6 | 31.3 |
| 27.0 | 13.3 |
| 27.4 | 15.1 |
| 27.7 | 24.7 |
| 28.6 | 22.4 |
| 31.3 | 16.5 |
| 32.0 | 16.5 |

The angle measurements are ±0.2° 2θ. Key defining peaks for solid-state Form A of anhydrous relugolix include one or more of 10.7, 20.9, and 19.2° 2θ. In one embodiment, key defining peaks for solid-state Form A of anhydrous relugolix include all of 10.7, 20.9, and 19.2° 2θ.

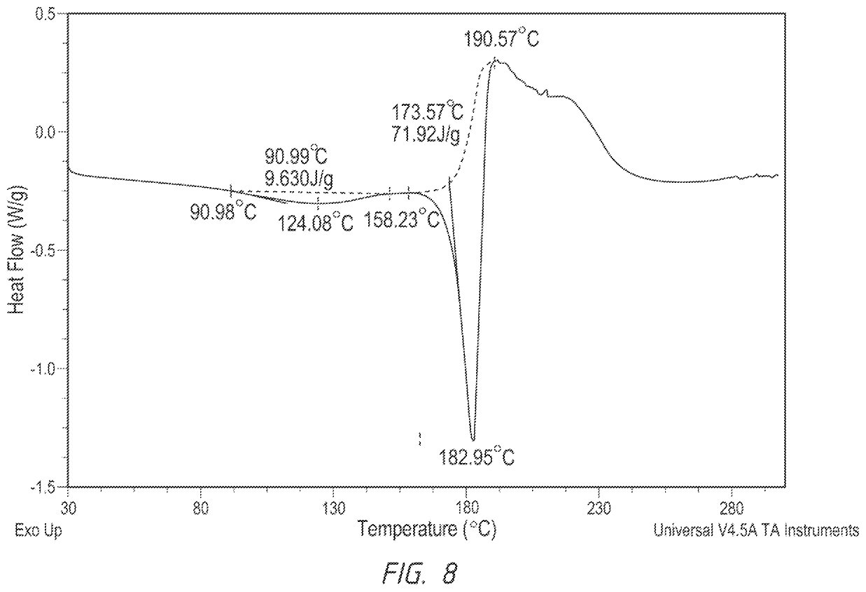

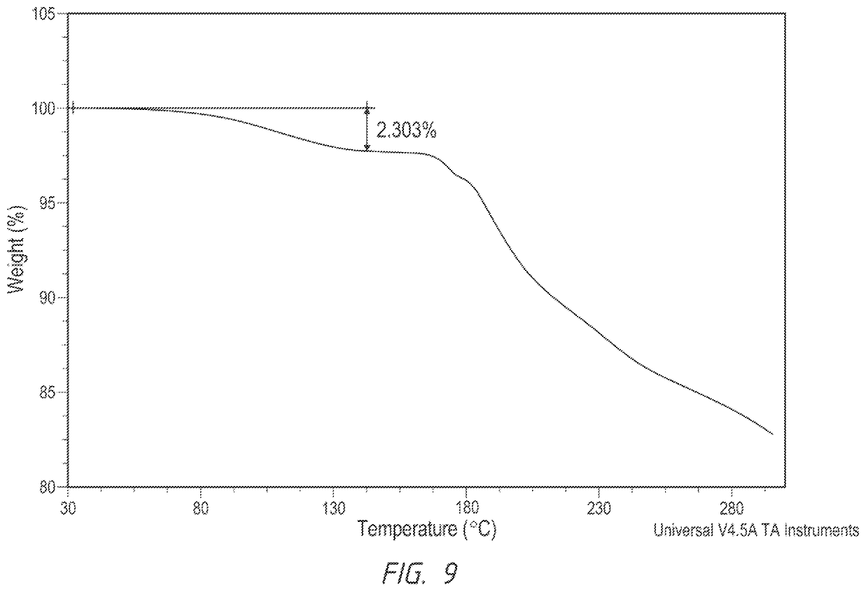

DSC analysis of Form A of anhydrous relugolix shows the start of an endothermic event at about 158° C. with an endothermic event at about 183° C., as depicted in FIG. 8. TGA analysis shows a loss of about 2.3 weight % up to about 140° C., as depicted in FIG. 9.

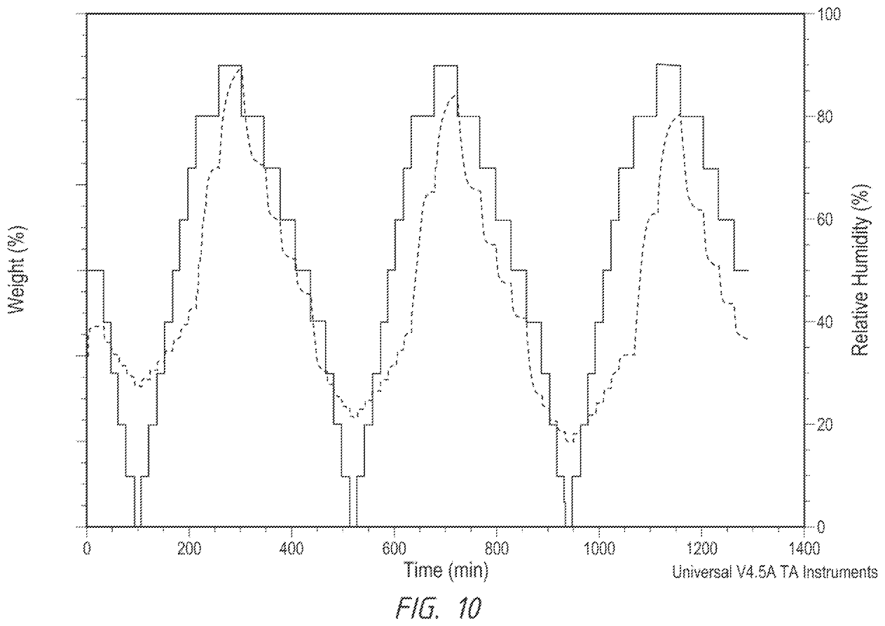

DVS analysis of Form A of anhydrous relugolix shows a weight % loss of about 2% when the sample is exposed to relative humidity levels from about 0 to 95%, as depicted in FIG. 10.

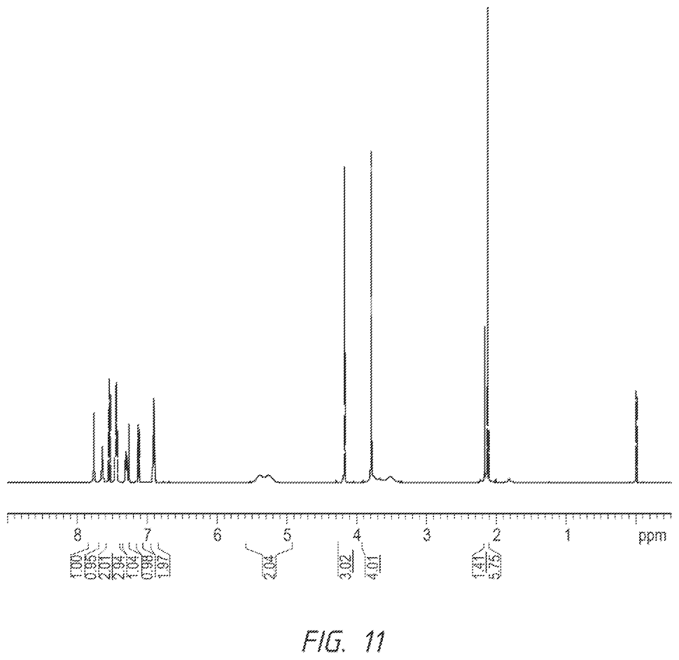

¹H NMR analysis indicates the presence of residual acetone at 2.13 ppm in Form A of anhydrous relugolix, as depicted in FIG. 11.

Form A of anhydrous relugolix remains stable at various humidity levels as evidenced by XRPD analysis after DVS. Also, XRPD shows no changes after drying the sample for about 18 hours at about 30° C. under vacuum.

### Example 3

### Preparation of Form B of Anhydrous Relugolix

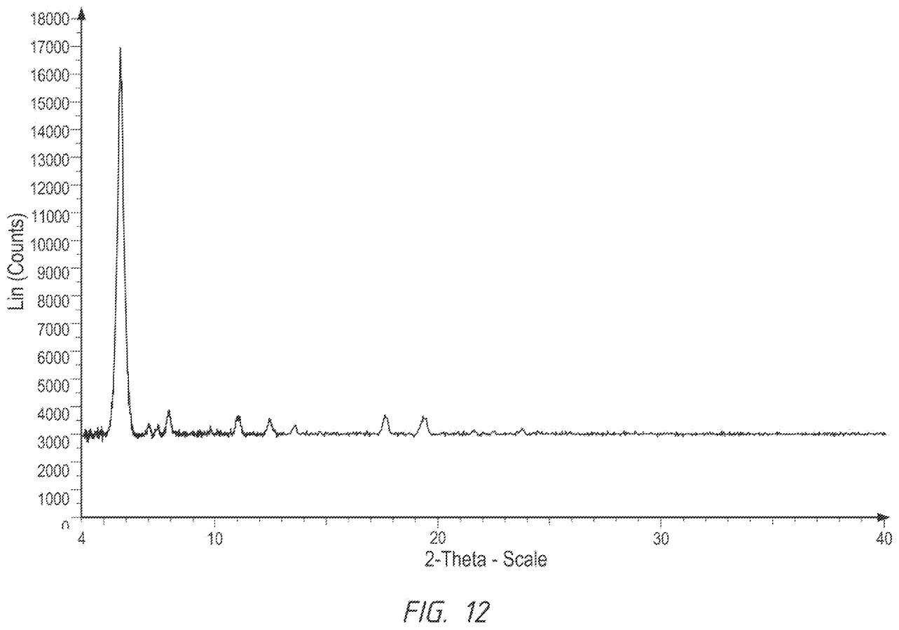

120 mL of DCM is added to 8.2 g of relugolix. The mixture is stirred for about 5 minutes, resulting in a slurry. About 100 mL of water is added to the slurry and stirred for about 15 minutes. After the stirring is stopped, some solids remain at the bottom of the flask and a bilayer is visible with a yellow organic bottom layer and mostly clear to hazy-clear aqueous layer on the top. The liquid is then decanted into a separatory funnel. 100 mL of DCM is added to the undissolved solids and stirred, forming a slurry. 100 mL of water is added to the slurry, stirred for about 15 minutes, and the liquid is decanted into the separatory funnel. 25 mL of DCM is added to any undissolved solids. The organic layer is vacuum filtered to remove any remaining solids. No drying agent is used to remove visible water droplets. The solvent in the organic layer is evaporated using a rotary evaporator at 35° C. and under a high vacuum pump for at least 3 hours. The isolated yellow solids (8.0 g, 97.6% yield) are identified as Form B of anhydrous relugolix. A representative XRPD pattern for Form B of anhydrous relugolix is shown in FIG. 12.

XRPD 2θ pattern peaks and relative % intensity values for the peaks of Form B of anhydrous relugolix are shown in Table 3.

TABLE 3
Average Peak List for Form B of
anhydrous relugolix diffractogram

| Angle (2θ°) | Intensity |
|---|---|
| degree | % |
| 5.7 | 100 |
| 7.0 | 20 |
| 7.4 | 19.6 |
| 7.9 | 22.5 |
| 9.8 | 19.3 |
| 11.0 | 21.7 |
| 12.4 | 21.1 |
| 13.5 | 19.6 |
| 17.6 | 21.6 |
| 19.4 | 21.3 |
| 21.6 | 18.5 |
| 23.7 | 19.1 |

The angle measurements are ±0.2° 2θ. A key defining peak for solid-state Form B of anhydrous relugolix includes 5.7° 2θ.

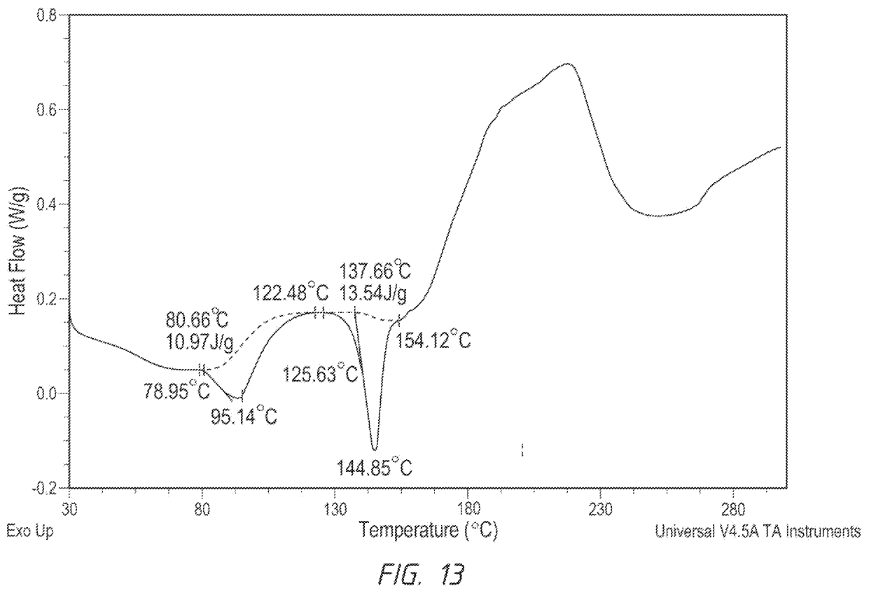

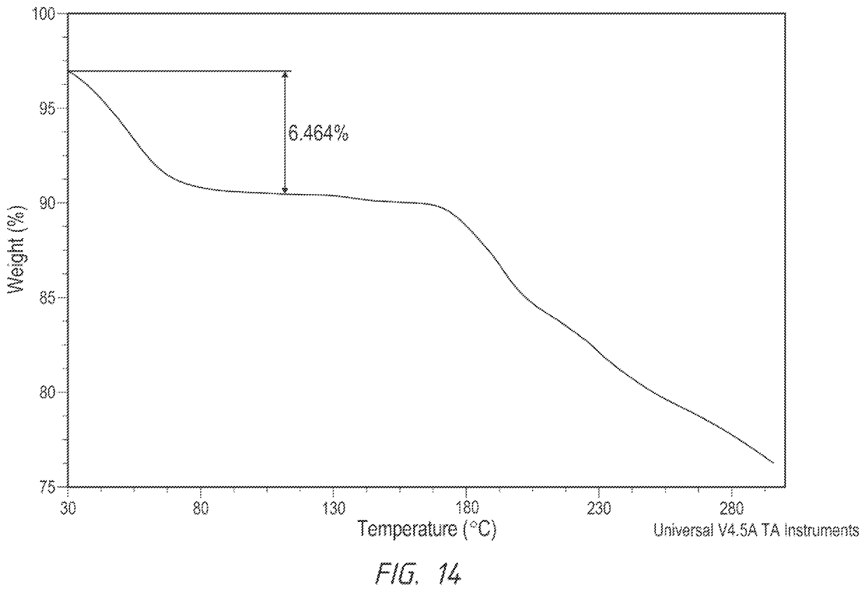

DSC analysis of Form B of anhydrous relugolix shows a loss of solvent at an onset temperature of about 79° C. and the onset of an endothermic event at about 126° C. with an endothermic event at about 145° C., as depicted in FIG. 13. TGA analysis shows a loss of greater than about 6 weight % up to about 105° C., as depicted in FIG. 14.

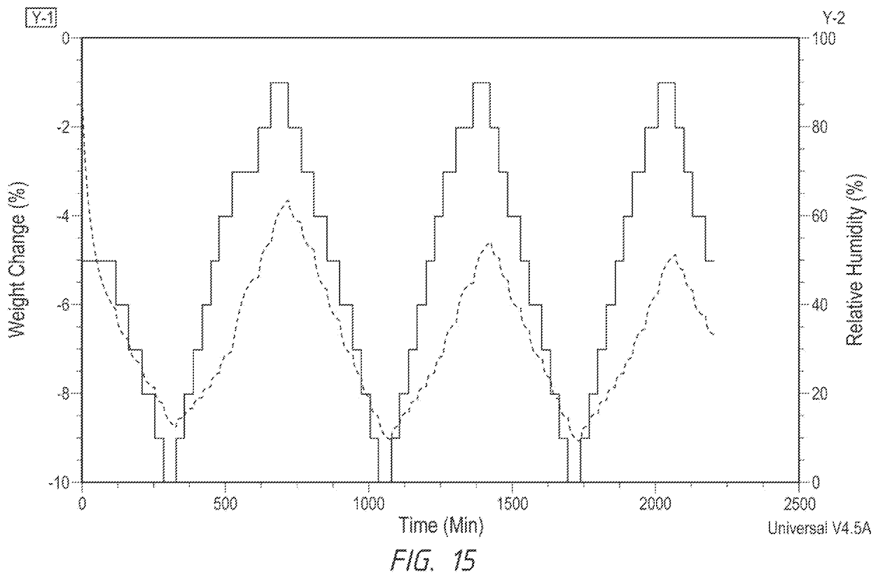

DVS analysis of Form B of anhydrous relugolix shows a weight loss of about 7% at relative humidity levels between about 0 to about 95%, as depicted in FIG. 15.

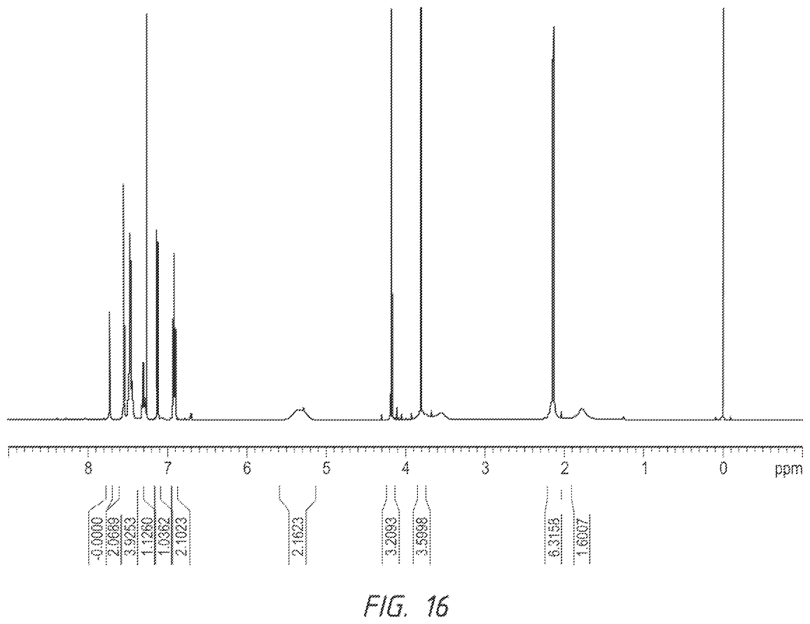

¹H NMR analysis of Form B of anhydrous relugolix confirms its structure and is depicted in FIG. 16.

Form B of anhydrous relugolix remains stable at various humidity levels, as evidenced by XRPD after DVS.

### Example 4

### Preparation of Form C of Anhydrous Relugolix

About 10 volumes of IPAc is added to Form B of anhydrous relugolix (weight(g_{relugolix}) to volume(mL_{IPAc})). The mixture is stirred overnight at ambient temperature resulting in a slurry. The slurry is decanted and the isolated material is dried in a vacuum oven at about 35-40° C. overnight and identified as Form C of anhydrous relugolix.

XRPD 2θ pattern peaks and relative % intensity values for the peaks of Form C of anhydrous relugolix are shown in Table 4.

TABLE 4
Average Peak List for Form C of
anhydrous relugolix diffractogram

| Angle (2θ°) | Intensity |
|---|---|
| degree | % |
| 6.8 | 62.1 |
| 7.7 | 34.6 |
| 8.3 | 100 |
| 10.0 | 14.6 |
| 11.8 | 9.9 |
| 13.1 | 15.1 |
| 13.6 | 13.4 |
| 14.4 | 10.3 |
| 15.6 | 11.3 |
| 18.0 | 10.7 |
| 19.9 | 28.7 |
| 20.8 | 10.1 |
| 21.5 | 14 |
| 22.3 | 16.3 |
| 24.9 | 24.9 |
| 26.2 | 19.8 |

The angle measurements are ±0.2° 2θ. Key defining peaks for solid-state Form C of anhydrous relugolix include one or more of 8.3, 6.8, 7.7, and 19.9° 2θ. In one embodiment, key defining peaks for solid-state Form C of anhydrous relugolix include all of 8.3, 6.8, 7.7, and 19.9° 2θ.

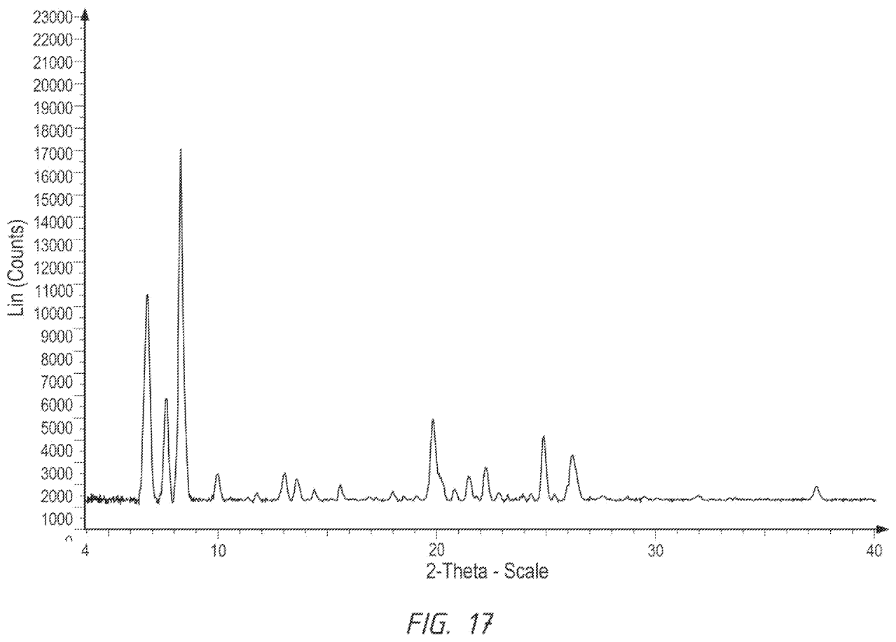

A representative XRPD pattern for Form C of anhydrous relugolix is shown in FIG. 17.

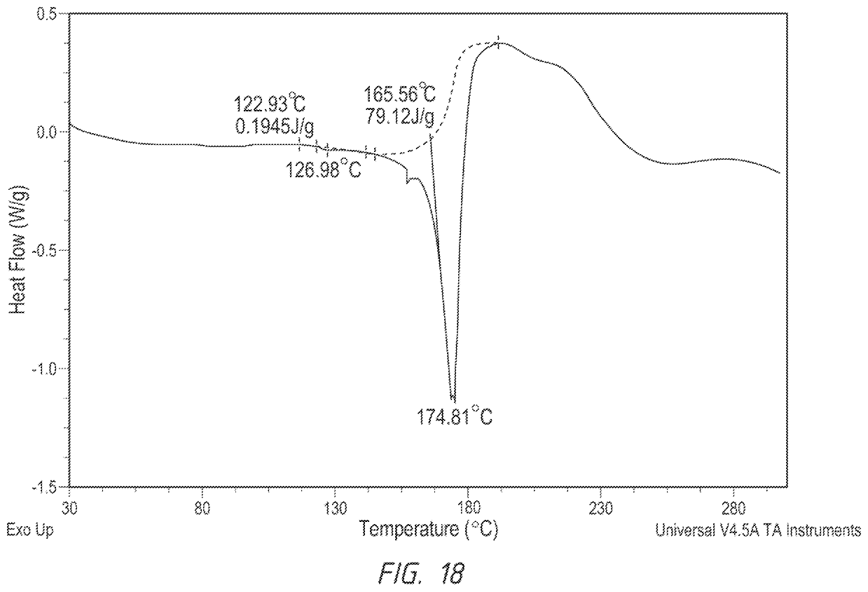

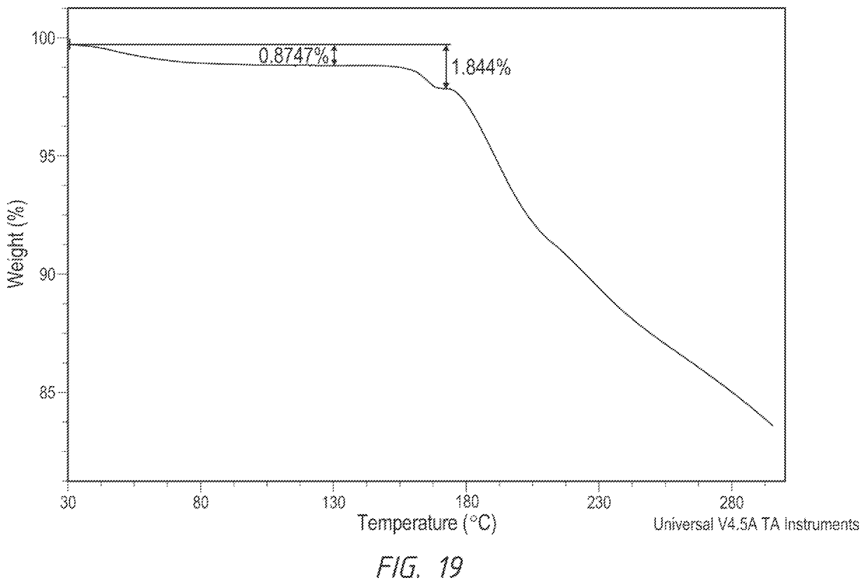

DSC analysis of Form C of anhydrous relugolix shows the onset of an endothermic event at about 140° C. with an endothermic event at about 175° C., as depicted in FIG. 18. TGA analysis shows less than about 1% weight loss up to about 143° C., as depicted in FIG. 19.

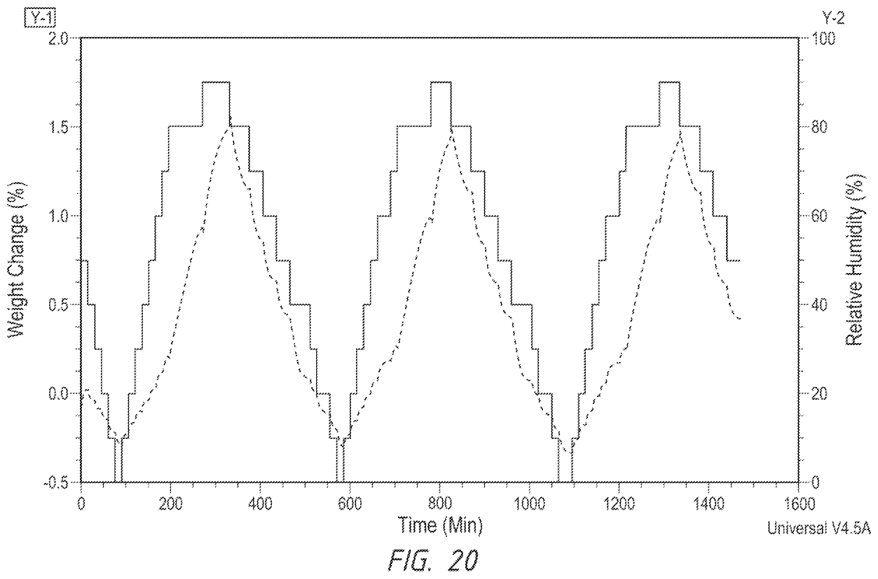

DVS analysis of Form C of anhydrous relugolix shows about a 2% water absorption and secretion of it all when the material is exposed to relative humidity between about 0 to about 95%, as depicted in FIG. 20.

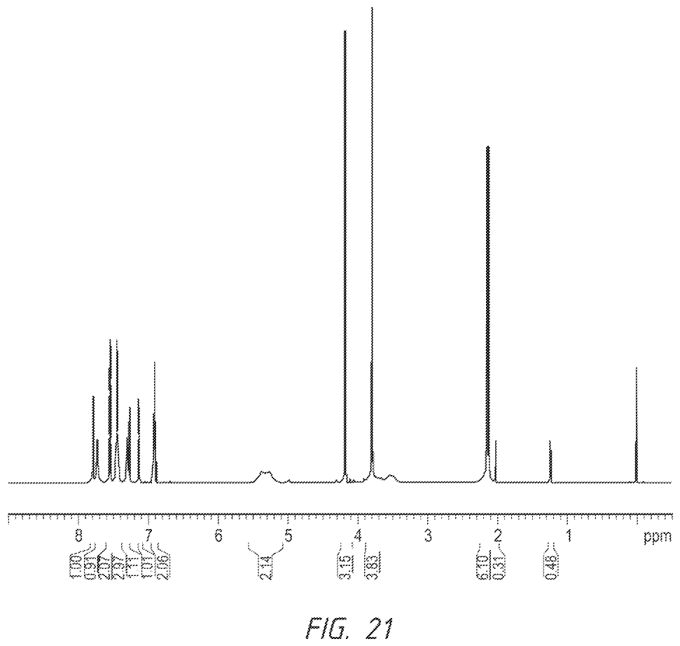

¹H NMR analysis indicates the presence of isopropyl acetate at 2 ppm (3H) which corresponds to about 1.6 weight %, as depicted in FIG. 21.

Form C of anhydrous relugolix remains stable at various humidity levels as evidenced by XRPD analysis after DVS.

The above examples are set forth to aid in the understanding of the disclosure and are not intended and should not be construed to limit in any way the disclosure set forth in the claims which follow hereafter.

1. A DMF solvate of relugolix.

2. The DMF solvate of claim 1 which is Form A of the DMF solvate of relugolix.

3. The DMF solvate according to claim 2, which is characterized by having at least 2 or more X-ray powder diffraction peaks selected from about 20.1, 24.3, and 9.0° 2θ±0.2° 2θ.

4. The DMF solvate according to claim 2, which is characterized by an onset of an endothermic event at about 99° C.±3° C., as measured by differential scanning calorimetry.

5. The DMF solvate according to claim 2, which is characterized by an endothermic event at about 149° C.±3° C., as measured by differential scanning calorimetry.

6. A process for the preparation of the DMF solvate according to claim 2 comprising:
a) mixing a solution of relugolix in DMF with an anti-solvent; and
b) stirring the mixture of step a) to yield Form A of the DMF solvate of relugolix as a precipitate.

7. The process according to claim 6, wherein the ratio of relugolix to DMF in the solution of relugolix in DMF is about 1:5 weight (g_{relugolix}) to volume (mL_{DMF}).

8. The process according to claim 6, wherein the anti-solvent is tert-butylmethyl ether or toluene.

9. The process according to claim 6, wherein the ratio of relugolix in the solution of relugolix in DMF to anti-solvent is from about 1:10 to about 1:13 weight (g_{relugolix}) to volume (mLₐₙₜᵢ₋ₛₒₗᵥₑₙₜ).

10. The process according to claim 6, wherein the stirring occurs for about 15-18 hours.

11. The process according to claim 6, wherein the stirring the mixture of step a) to produce a precipitate occurs at ambient temperature.

12. The DMF solvate according to claim 2, which has single crystal parameters
a=11.1Å±1.5%
b=12.0Å±1.5%
c=14.0Å±1.5%
α=112°±3°
β=110°±3°
γ=91°±3°

13. The DMF solvate according to claim 2, which has a cell volume of about 1609Å³±3%.

14. Form A of anhydrous relugolix.

15. Form A of anhydrous relugolix according to claim 14, which is characterized by having X-ray powder diffraction peaks selected from about 10.7, 20.9, and 19.2° 2θ±0.2° 2θ.

16. Form A of anhydrous relugolix according to claim 14, which is characterized by an onset of an endothermic event at about 158° C.±3° C., as measured by differential scanning calorimetry.

17. Form A of anhydrous relugolix according to claim 14, which is characterized by an endothermic event at about 183° C.±3° C., as measured by differential scanning calorimetry.

18. A process for the preparation of Form A of anhydrous relugolix according to claim 14 comprising:
a) forming a solution of relugolix in acetone wherein the relugolix is in about 10 volumes of acetone (weight(g_{relugolix}):volume(mL_{acetone})); and
b) stirring the solution of relugolix in acetone to yield Form A of anhydrous relugolix as a precipitate.

19. A process for the preparation of Form B of anhydrous relugolix which is characterized by having an X-ray powder diffraction peak at about 5.7°2θ±0.2° 2θ comprising:
a) forming a solution of relugolix in DCM wherein the relugolix is in about 20 volumes of DCM (weight(g_{relugolix}):volume(mL_{DCM})); and
b) evaporating the DCM to yield Form B of anhydrous relugolix.

20. A process for the preparation of Form B of anhydrous relugolix which is characterized by having an X-ray powder diffraction peak at about 5.7°2θ±0.2°2θ comprising:
a) mixing a solution of relugolix in DCM wherein the relugolix is in at least about 20 volumes of DCM (weight(g_{relugolix}):volume(mL_{DCM}) with an anti-solvent wherein the anti-solvent is at about a 1:1 ratio of anti-solvent to DCM (volumeₐₙₜᵢ₋ₛₒₗᵥₑₙₜ:volume_{DCM});
b) stirring the mixture of step a) for a period of time to yield Form B of anhydrous relugolix as a precipitate.

21. The process according to claim 20 wherein the anti-solvent is cumene, cyclohexane, TBME, heptane, or toluene.

22. Form C of anhydrous relugolix.

23. Form C of anhydrous relugolix according to claim 22, which is characterized by having X-ray powder diffraction peaks selected from about 8.3, 6.8, 7.7, and 19.9° 2θ±0.2°2θ.

24. Form C of anhydrous relugolix according to claim 22, which is characterized by an onset of an endothermic event at about 140° C.±3° C., as measured by differential scanning calorimetry.

25. Form C of anhydrous relugolix according to claim 22, which is characterized by an endothermic event at about 175° C.±3° C., as measured by differential scanning calorimetry.

26. A process for the preparation of Form C of anhydrous relugolix according to claim 22 comprising;
a) adding about 10 volumes of an organic solvent to Form B of anhydrous relugolix (weight(g_{relugolix}):volume(mL_{organic solvent}); and
b) stirring the mixture of organic solvent and Form B of anhydrous relugolix for about 16-24 hours resulting in a slurry of Form C of anhydrous relugolix.

27. The process according to claim 26, wherein the organic solvent is isopropyl acetate or 2-butanol.

28. A pharmaceutical composition comprising a pharmaceutically effective amount of a compound selected from the DMF solvate of relugolix according to claim 1, Form A of anhydrous relugolix according to claim 14, and Form C of anhydrous relugolix according to claim 22, and a pharmaceutically acceptable excipient.

29. A method of treating disease in a patient comprising administering the pharmaceutical composition according to claim 28 to a patient in need thereof.

30. The method of treating disease according to claim 29, wherein the disease is uterine fibroids, endometriosis, or prostate cancer.
